(19) United States
(12) Patent Application Publication
(10) Pub. No.: US 2025/0303429 A1
(43) Pub. Date: Oct. 2, 2025

(54) DEVICE FOR DISPENSING A FLUID PRODUCT AND METHOD FOR PRIMING SAME

(71) Applicant: APTAR FRANCE SAS, Le Neubourg (FR)

(72) Inventor: Ludovic PETIT, Vitot (FR)

(73) Assignee: APTAR FRANCE SAS, Le Neubourg (FR)

(21) Appl. No.: 18/861,024

(22) PCT Filed: Apr. 28, 2023
(86) PCT No.: PCT/FR2023/000065

§ 371 Date: Oct. 28, 2024

(30) Foreign Application Priority Data
Apr. 29, 2022 (FR) FR2204063

(51) Int. Cl. B05B 11/10; A61M 15/08

(52) U.S. Cl. CPC B05B 11/1061; A61M 15/08; B05B 11/1001; B05B 11/1047

(57) ABSTRACT

Device having a dispenser on a reservoir containing one or more doses of fluid product. The dispenser has a pump body containing a chamber and a head with an orifice with a removable cap. The head is axially displaceable with respect to the pump body. A piston cohesive with the head is adapted to slide in the chamber between a high abutment position, in which the volume of the chamber is maximal, and a low abutment position, in which the volume of the chamber is minimal. During fitting of the dispenser, the protective cap tightly closes the dispensing orifice and the piston is in its low abutment position. The chamber is filled with the fluid product by displacing the piston from its low abutment position towards its high abutment position, and the fluid product is dispensed by displacing the piston from its high abutment position towards its low abutment position.

The present invention relates to a device for dispensing a fluid product, in particular for nasal delivery of one or two doses of a pharmaceutical fluid product, and to a method for priming it.

A disadvantage of certain devices for nasal delivery of fluid product, in particular those using a pump with a dosing chamber in order to define the volume of the dose dispensed upon each actuation, concerns the need to prime the pump in order to replace the air initially contained in the dosing chamber with fluid. This priming is generally accomplished by one or more actuations of the device, with a possible loss of fluid product as a consequence.

The objective of the present invention is to provide a device for dispensing fluid product which does not reproduce the aforementioned disadvantages.

More particularly, the objective of the present invention is to provide such a device which does not require priming.

Another objective of the present invention is to provide such a device that is simple and inexpensive to manufacture and to assemble.

The present invention therefore concerns a device for dispensing fluid product, comprising a dispenser fitted on a reservoir containing one or more doses of fluid product, said dispenser comprising a pump body containing a dosing chamber, and a dispensing head comprising a dispensing orifice provided with a removable protective cap forming a closure for said dispensing orifice, said dispensing head being axially displaceable with respect to said pump body, a piston, which is cohesive with said dispensing head, being adapted to slide in said dosing chamber between a high abutment position, in which the volume of said dosing chamber is maximal, and a low abutment position, in which the volume of said dosing chamber is minimal, in which, during the fitting of said dispenser on said reservoir, said protective cap is positioned for tightly closing said dispensing orifice and said piston is in its low abutment position, said piston being adapted to slide only once from its low abutment position towards its high abutment position in order to fill said dosing chamber with the fluid product from said reservoir, and said piston being adapted, after removal of said protective cap, to slide only once from its high abutment position towards its low abutment position in order to dispense said fluid product through said dispensing orifice in one or more doses.

Advantageously, a hollow rod is disposed in said dispensing head, said hollow rod defining a first portion of an expulsion channel connecting said dosing chamber to said dispensing orifice during the actuation, said hollow rod comprising said piston.

Advantageously, an insert is disposed in the dispensing head between said dispensing orifice and said hollow rod, in order to define a second portion of the expulsion channel upstream of said dispensing orifice.

Advantageously, said dosing chamber comprises, on the lower axial side, an opening connected to said reservoir, in particular via a dip tube, said opening being closed by a closure element forming a valve which opens during filling of said dosing chamber and which closes from the start of each actuation.

Advantageously, said reservoir contains at least two doses of fluid product dispensed in successive actuations, said device comprising dose separation means for stopping the actuating stroke of said piston before its low abutment position.

In accordance with a first advantageous variation, said dispensing head comprises at least one internal radial projection, and said pump body and/or an element which is cohesive with said pump body comprises at least one external radial projection, each internal radial projection axially abutting against a respective external radial projection after dispensing the first dose, such that a rotation between said radial projections is necessary in order to be able to dispense another dose.

According to a second advantageous variation, said dispensing head comprises at least one internal radial projection, said dose separation means comprising a removable ring radially disposed around said pump body and/or around an element which is cohesive with said pump body, each internal radial projection axially abutting against said removable ring after dispensing the first dose, such that a radial removal of said removable ring is necessary in order to be able to dispense another dose.

According to a third advantageous variation, said dose separation means comprises a removable collar, disposed axially in said dispensing head, a lower edge of said removable ring axially abutting against said pump body and/or against an element which is cohesive with said pump body after dispensing the first dose, such that a removal of said removable collar is necessary in order to be able to dispense another dose.

Advantageously, said pump body comprises at least one axial extension which, after dispensing the last dose, protrudes axially through said dispensing head, thereby forming an end-of-use indicator.

Advantageously, said removable protective cap is connected to said dispensing head in a captive manner by a connector.

Advantageously, said removable protective cap comprises a first use indicator, secured to said dispensing head, and which is detached from said removable protective cap when the latter is removed from said dispensing head.

Advantageously, said removable protective cap comprises at least one axial tab which, in the closed position, penetrates into the interior of said dispensing head, each axial tab comprising a snap-fit projection cooperating with a portion of said dispensing head in order to prevent the removal of said cap, each axial tab being locked in its snap-fitted position by an axial extension of said pump body, which prevents any deformation of said axial tab, such that when said dosing chamber is filled, locking of said axial tab by said axial extension is eliminated by axial displacement of said dispensing head relative to said pump body, enabling said cap to be removed.

The present invention also provides a method for priming a device for dispensing fluid product, comprising the following steps:
providing a reservoir containing at least one dose of fluid product,
providing a dispenser comprising a pump body containing a dosing chamber, and a dispensing head comprising a dispensing orifice provided with a removable protective cap forming a closure, said dispensing head being axially displaceable with respect to said pump body, a piston, which is cohesive with said dispensing head, being adapted to slide in said dosing chamber between a high abutment position in which the volume of said dosing chamber is maximal, and a low abutment position, in which the volume of said dosing chamber is minimal,
   said method comprising the following steps:
fitting said dispenser on said reservoir, by inserting said pump body into said reservoir with said removable protective cap in the closed position and said piston in the low abutment position,
axially displacing said piston only once between its low abutment position and its high abutment position, in order to fill said dosing chamber from said reservoir,
removing said removable protective cap, and
axially displacing said piston only once between its high abutment position and its low abutment position, in order to dispense one dose or more doses of fluid product through said dispensing orifice.

Advantageously, said reservoir contains at least two doses of fluid product which are loaded into the dosing chamber during the axial displacement of said piston between its low abutment position and its high abutment position, said device comprising dose separation means for stopping the actuating stroke of said piston before said low abutment position, in order to dispense said fluid product in at least two doses.

These features and advantages and others of the present invention will become more apparent from the following detailed description, made with reference to the accompanying drawings, given by way of non-limiting examples, and in which:

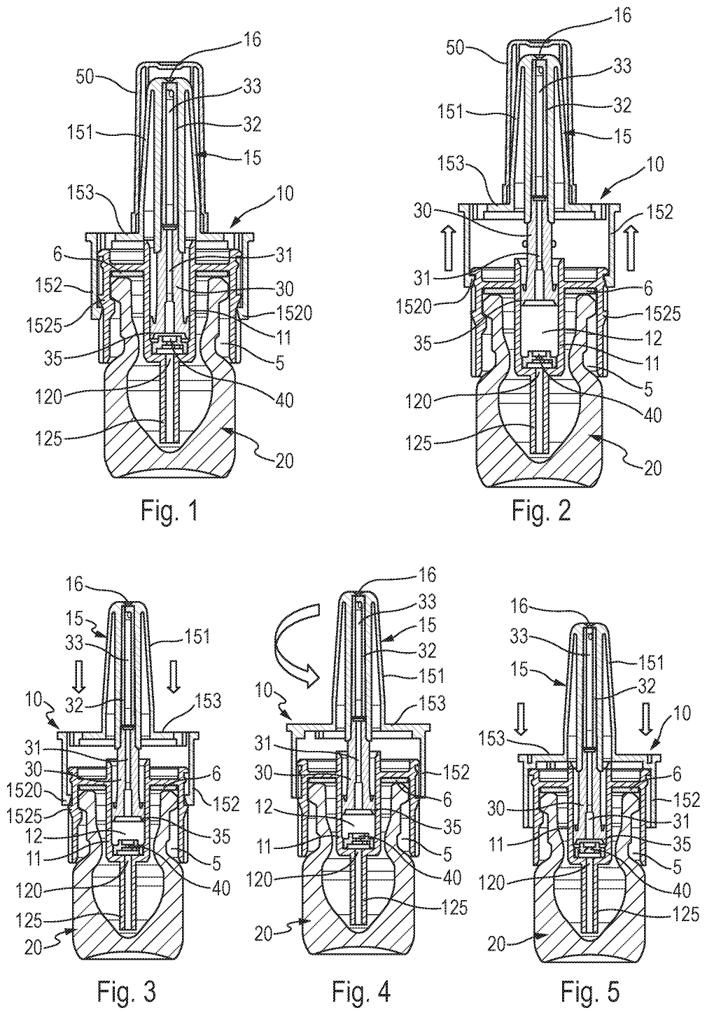

FIG. 1 is a schematic, cross-sectional view of a device for dispensing fluid product in accordance with a first embodiment, after fitting the dispenser on the reservoir,

FIG. 2 is a view similar to that of FIG. 1, after loading fluid product into the pump chamber,

FIG. 3 is a view similar to that of FIG. 2, after dispensing the first dose,

FIG. 4 is a view similar to that of FIG. 3, before dispensing the second dose,

FIG. 5 is a view similar to that of FIG. 4, after dispensing the second dose,

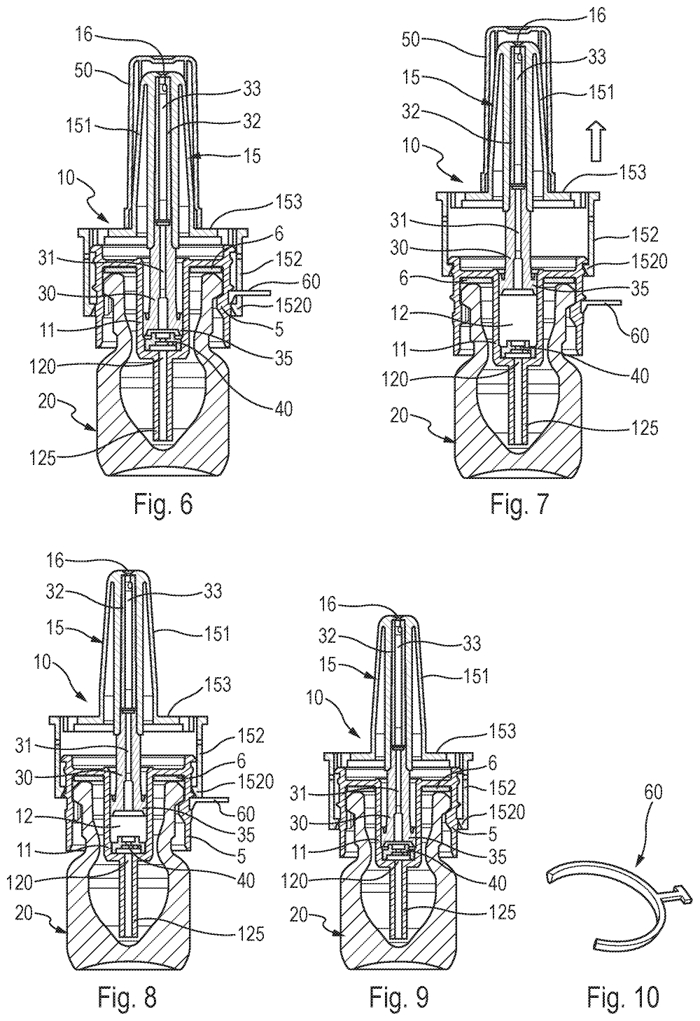

FIG. 6 is a schematic cross-sectional view of a device for dispensing fluid product in accordance with an advantageous second embodiment, after fitting the dispenser on the reservoir,

FIG. 7 is a view similar to that of FIG. 6, after loading fluid product into the pump chamber,

FIG. 8 is a view similar to that of FIG. 7, after dispensing the first dose,

FIG. 9 is a view similar to that of FIG. 8, after dispensing the second dose,

FIG. 10 is a schematic perspective view of the dose separation element of the second embodiment,

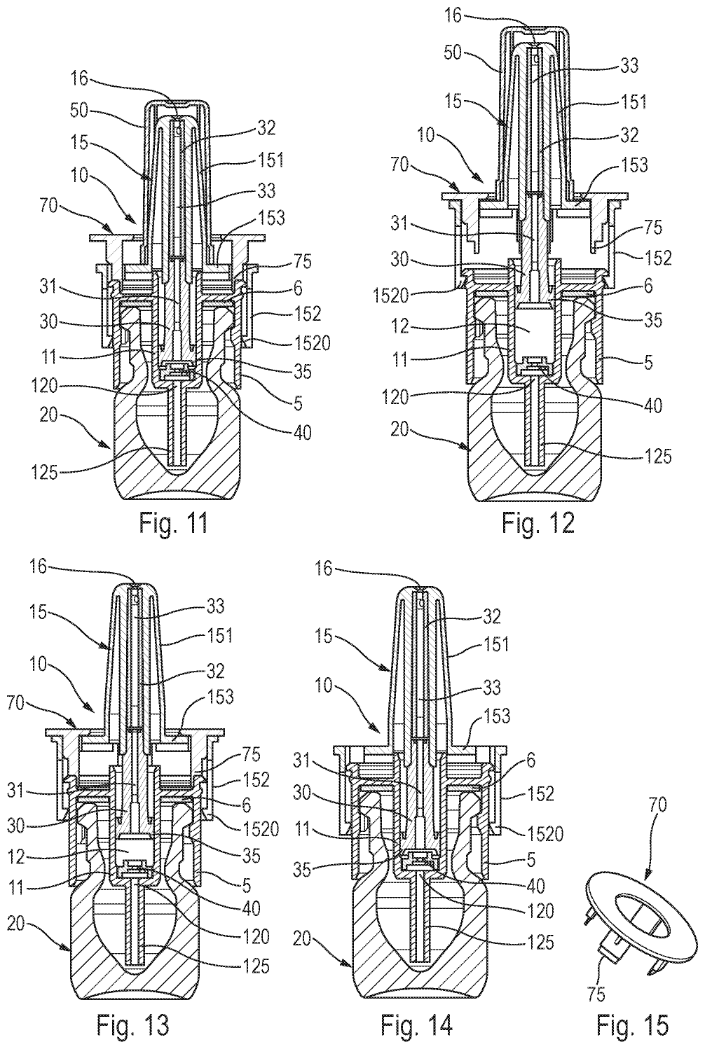

FIG. 11 is a schematic cross-sectional view of a device for dispensing fluid product in accordance with an advantageous third embodiment, after fitting the dispenser on the reservoir,

FIG. 12 is a view similar to that of FIG. 11, after loading fluid product into the pump chamber,

FIG. 13 is a view similar to that of FIG. 12, after dispensing the first dose,

FIG. 14 is a view similar to that of FIG. 13, after dispensing the second dose,

FIG. 15 is a schematic perspective view of the dose separation element of the second embodiment,

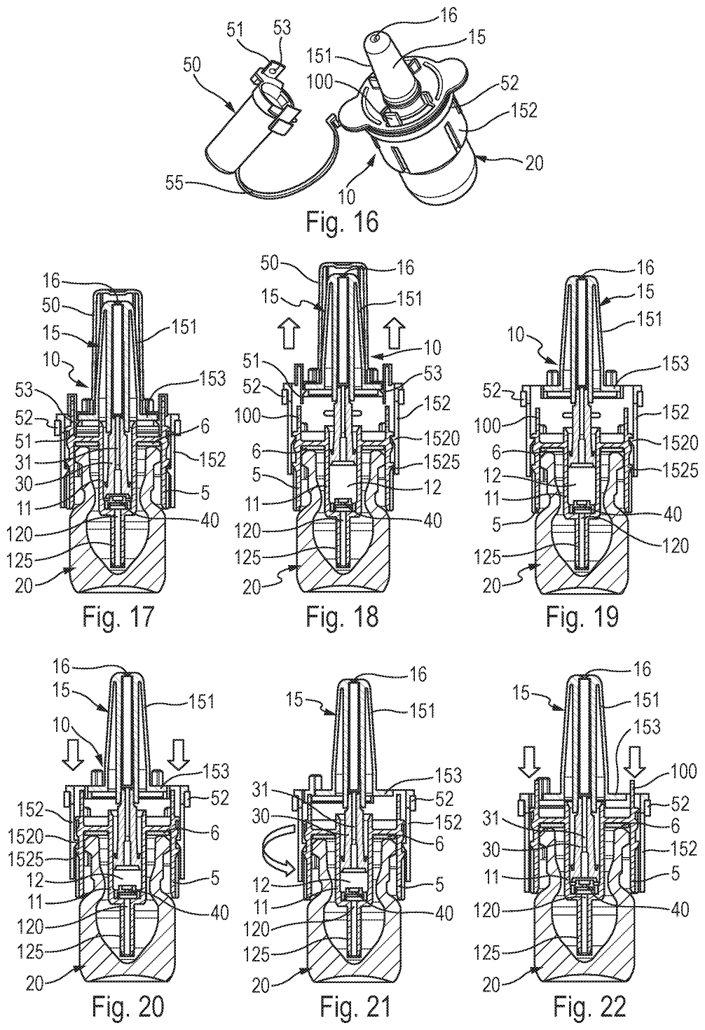

FIG. 16 is a diagrammatic perspective view of a device for dispensing fluid product in accordance with a fourth advantageous embodiment,

FIG. 17 is a schematic cross-sectional view of a device for dispensing fluid product in accordance with an advantageous fifth embodiment, after fitting the dispenser on the reservoir,

FIG. 18 is a view similar to that of FIG. 17, after loading fluid product into the pump chamber,

FIG. 19 is a view similar to that of FIG. 18, after removing the removable protective cap,

FIG. 20 is a view similar to that of FIG. 19, after dispensing the first dose,

FIG. 21 is a view similar to that of FIG. 20, before dispensing the second dose, and

FIG. 22 is a view similar to that of FIG. 21, after dispensing the second dose.

The terms “axial” and “radial” refer to the longitudinal central axis of the device. The terms “upstream” and “downstream” refer to the direction of flow of the fluid during actuation. The terms “high”, “low”, “upper” and “lower” refer to the upright position of the device represented in FIGS. 1 to 9, 11 to 14, and 17 to 22.

The present invention pertains to a single-dose device which contains only a single dose of fluid product in the reservoir 20, but it is particularly suitable for what are known as two-dose devices, in which the reservoir 20 contains two doses of fluid product, dispensed in two successive actuations. The various examples shown in the drawings relate to two-dose devices. Optionally, the reservoir 20 could contain more than two doses, for example three or four doses.

In accordance with the invention, the device comprises a dispenser 10 fitted on a reservoir 20 containing fluid product to be dispensed, typically a pharmaceutical type liquid.

The reservoir 20 is advantageously formed by a hollow and blind body, comprising a single opening and containing one dose or more doses of fluid product to be dispensed. In the exemplary embodiments shown in the figures, the reservoir 20 contains two doses of fluid product.

The dispenser 10 comprises a pump body 11 containing a dosing chamber 12, and a dispensing head 15 comprising a dispensing orifice 16, which is preferably axially oriented. This dispensing orifice 16 serves to dispense a dose of fluid product out of said dispensing head 15 during the actuation of the device by a user. The dispensing head 15 is axially displaceable with respect to the pump body 11.

At rest, a removable protective cap 50 is disposed on the dispensing head 15 in order to tightly close the dispensing orifice 16. This protective cap 50, when it is positioned on the dispensing head 15, therefore forms a closure for the dosing chamber 12, isolating it from the atmosphere.

The dispensing head 15 comprises an extended nasal endpiece 151 comprising said dispensing orifice 16 at its axial end, as well as a lateral body 152, connected to said nasal endpiece 151 via an approximately radial flange 153.

A hollow rod 30 is connected to the dispensing head 15, said hollow rod 30 defining a first expulsion channel portion 31.

An insert 32, which is advantageously hollow, may be disposed in the dispensing head 15 in order to define a second portion 33 of expulsion channel upstream of the dispensing orifice 16. This insert not only enables the dead volume of this second expulsion channel portion 33 to be limited, but also permits spraying through the dispensing orifice 16 to be carried out correctly.

Advantageously, a spray profile may be provided directly upstream of said dispensing orifice 16, between said insert 32 and the bottom wall of the dispensing head 15.

On the opposite side, the hollow rod 30 comprises a piston 35 adapted to slide in the dosing chamber 12 of the pump body 11 between a high abutment position, also termed the top dead center, and a low abutment position, also termed the bottom dead center. This piston 35 may be formed in one piece with the hollow rod 30. In a variation, a seal, in particular an O-ring, may be fitted on the hollow rod 30 in order to form the piston. Other known variations for producing the piston 35 may also be envisaged.

The piston 35 is adapted to slide only once between its low abutment position and its high abutment position, in order to fill the dosing chamber 12, and only once between its high abutment position and its low abutment position, in order to dispense the contents of the dosing chamber 12, in one dose or more doses.

The dispenser 10 may be cohesive with the reservoir 20 in any known manner, for example by using a securing ring 5 in order to secure the pump body 11 on a neck of the reservoir. Advantageously, a neck seal 6 is interposed between the securing ring 5 and the reservoir 20. Preferably, as shown in the drawings, the securing ring 5 is produced from a part which is in one piece with the pump body 11.

The dosing chamber 12 is open on its two axial sides. On the upper axial side, it is the piston 35, the hollow rod 30 and the removable protective cap 50 that close the dosing chamber 12. On the lower axial side, the dosing chamber 12 comprises an opening 120 connected to the reservoir 20, advantageously via a dip tube 125. This opening 120 is closed by a closure element 40 forming a valve which opens during filling of the dosing chamber 12 and which closes from the start of each actuation. This valve 40 may be produced in any known manner, for example in the form of a floating seal, a split membrane or a ball.

This opening 120 together with the closure member 40 therefore forms a conventional inlet valve for the dosing chamber 12, enabling it to be filled due to the partial vacuum generated by the piston 35 which rises towards its rest position.

Thus, the invention makes it possible to produce a device without priming.

When the dispenser 10 is fitted on the reservoir 20, the piston 35 is positioned in the low abutment position with respect to the pump body 11, and the protective cap 50 is positioned on the dispensing head 15 in the closed position in order to tightly close the dispensing orifice 16. The dosing chamber 12 therefore has a minimal volume after assembly, as can be seen in FIG. 1, with the piston 35 being substantially in contact with the closure element 40. Only the dead volume formed by the first and second expulsion channel portions 31, 33 contains air.

Before the first actuation, the user manually displaces the dispensing head 15 axially away from the reservoir 20, in order to bring the piston 35 into its high abutment position shown in FIG. 2. During this axial displacement, the closure element 40 forming a valve opens under the partial vacuum generated by the displacement of the piston 35, and the fluid product contained in the reservoir is sucked into the dosing chamber 12. In this high abutment position of the piston, the volume of the dosing chamber 12 is maximal.

During the first actuation, the user removes the protective cap 50, places two fingers on the radial flange 153 formed on the dispensing head 15, and presses with the thumb on the bottom of the reservoir 20. During such an actuation, the reservoir 20 is therefore pushed axially in the direction of the dispensing orifice 16, such that the piston 35 slides in the dosing chamber 12. The opening 120 is closed by the pressure exerted by the fluid product on the closure member 40 forming a valve. The fluid product contained in the dosing chamber 12 is therefore dispensed through the expulsion channel 31, 33 in the direction of the dispensing orifice 16.

If the device is a single-dose device, the entirety of the fluid product contained in the dosing chamber 12 is dispensed in a single actuation.

If the device is a two-dose or a multi-dose device, the device comprises dose separation means for stopping the actuating stroke of the piston before the bottom dead center. Thus, if the device is a two-dose device, the actuating stroke of the piston 35 is stopped approximately half-way, so that half of the fluid product contained in the dosing chamber 12 is dispensed during the first actuation, and the other half during a second actuation.

Advantageously, the first actuating stroke corresponding to the first dose is slightly greater than the actuating strokes for the other doses, in order to take into account the dead volume formed by the expulsion channel 31, 33, which is filled with air before the first actuation.

In the first embodiment of FIGS. 1 to 5, the dose separation means necessitates a rotation of the dispensing head with respect to the reservoir 20 and/or the securing ring 5. Thus, the lateral body 152 of the dispensing head 15 comprises at least one internal radial projection 1520, and the pump body 11 and/or the securing ring 5 comprises at least one external radial projection 1525, said internal radial projection 1520 coming into axial abutment against said external radial projection 1525 after dispensing the first dose. A rotation between these two radial projections 1520, 1525 is then necessary in order to be able to dispense another dose. Advantageously, there may be two diametrically opposed pairs of radial projections 1520, 1525.

In the second embodiment of FIGS. 6 to 10, the dose separation means comprises a removable ring 60 radially disposed around the pump body 11 and/or the securing ring 5. The internal radial projection 1520 of the dispensing head 15 comes into axial abutment against said removable ring 60 after dispensing the first dose. The radial removal of the removable ring 60 is then necessary in order to be able to dispense another dose.

FIG. 10 shows an example of a removable ring 60 in detail.

In the third embodiment of FIGS. 11 to 15, the dose separation means comprises a removable collar 70, disposed axially in the dispensing head 15, in particular in the radial flange 153. A lower edge 75 of said removable collar 70 comes into axial abutment against the pump body 11 and/or the securing ring 5 after dispensing the first dose. Removal of the removable collar 70 is then necessary in order to be able to dispense another dose.

FIG. 15 shows an example of a removable collar 70 in detail.

FIG. 16 shows a fourth embodiment, in which the removable protective cap 50 is connected to the dispensing head 15 in a captive manner by a connector 55. This implementation is applicable to all of the other embodiments of the invention.

In addition, the protective cap may comprise a first use indicator 52, secured to the dispensing head 15, and which is detached from the cap 50 when the cap is removed from said head. In a known manner, this first use indicator may be connected to the cap by severable bridges that break when the cap 50 is removed. This implementation is also applicable to all of the other embodiments of the invention.

In addition, the pump body 11 comprises at least one axial extension 100 which, after dispensing the last dose, protrudes axially through the radial flange 153 of the dispensing head 15, thereby forming an end-of-use indicator.

The fifth embodiment of FIGS. 17 to 22 is similar to the first embodiment of FIGS. 1 to 5, and only the differences will be described below.

A first difference concerns the cap 50, which cannot be removed from the dispensing head 15 until the dosing chamber 12 has been filled. This may advantageously be accomplished by at least one axial tab 51 of the cap 50 which, in the closed position, penetrates into the interior of the dispensing head 15. Each axial tab 51 comprises a snap-fit projection 53 that cooperates with a portion of the dispensing head 15, in particular the radial flange 153, in order to prevent removal. Each axial tab 51 is locked in its snap-fitted position by an axial extension 100 of the pump body 11, which prevents any deformation of said axial tab 51. When the dosing chamber 12 has been filled by axial displacement of the dispensing head 15 with respect to the pump body 11, locking of the axial tab 51 by the axial extension 100 is eliminated, as can be seen in FIG. 18. The cap 50 can then be removed from the dispensing head 15, by axial traction, which deforms the axial tab 51 in order to enable the snap-fitted projection to come apart.

Advantageously, after dispensing the last dose, said axial extension 100 forms the end-of-use indicator described with reference to FIG. 16.

Clearly, variations of the embodiments may be envisaged without departing from the scope of the present invention as defined in the accompanying claims.

1. A device for dispensing fluid product, comprising a dispenser fitted on a reservoir containing one or more doses of fluid product, said dispenser comprising a pump body containing a dosing chamber, and a dispensing head comprising a dispensing orifice provided with a removable protective cap forming a closure for said dispensing orifice, said dispensing head being axially displaceable with respect to said pump body, a piston, which is cohesive with said dispensing head, being adapted to slide in said dosing chamber between a high abutment position, in which the volume of said dosing chamber is maximal, and a low abutment position, in which the volume of said dosing chamber is minimal, characterized in that during the fitting of said dispenser on said reservoir, said protective cap is positioned for tightly closing said dispensing orifice and said piston is in its low abutment position, said piston being adapted to slide only once from its low abutment position towards its high abutment position in order to fill said dosing chamber with the fluid product from said reservoir, and said piston being adapted, after removal of said protective cap, to slide only once from its high abutment position towards its low abutment position in order to dispense said fluid product through said dispensing orifice in one or more doses.

2. The device as claimed in claim 1, in which a hollow rod is disposed in said dispensing head, said hollow rod defining a first portion of an expulsion channel connecting said dosing chamber to said dispensing orifice during the actuation, said hollow rod comprising said piston.

3. The device as claimed in claim 2, in which an insert is disposed in the dispensing head between said dispensing orifice and said hollow rod, in order to define a second portion of expulsion channel upstream of the dispensing orifice.

4. The device as claimed in claim 1, in which said dosing chamber comprises, on the lower axial side, an opening connected to said reservoir, in particular via a dip tube, said opening being closed by a closure element forming a valve which opens during filling of said dosing chamber and which closes from the start of each actuation.

5. The device as claimed in claim 1, in which said reservoir contains at least two doses of fluid product dispensed in successive actuations, said device comprising dose separation means for stopping the actuating stroke of said piston before its low abutment position.

6. The device as claimed in claim 5, in which said dispensing head comprises at least one internal radial projection, and said pump body and/or an element which is cohesive with said pump body comprises at least one external radial projection, each internal radial projection axially abutting against a respective external radial projection after dispensing the first dose, such that a rotation between said radial projections is necessary in order to be able to dispense another dose.

7. The device as claimed in claim 5, in which said dispensing head comprises at least one internal radial projection, said dose separation means comprising a removable ring radially disposed around said pump body and/or around an element which is cohesive with said pump body, each internal radial projection axially abutting against said removable ring after dispensing the first dose, such that a radial removal of said removable ring is necessary in order to be able to dispense another dose.

8. The device as claimed in claim 5, in which said dose separation means comprises a removable collar, disposed axially in said dispensing head, a lower edge of said removable collar axially abutting against said pump body and/or against an element which is cohesive with said pump body after dispensing the first dose, such that a removal of said removable collar is necessary in order to be able to dispense another dose.

9. The device as claimed in claim 1, in which said pump body comprises at least one axial extension which, after dispensing the last dose, protrudes axially through said dispensing head, thereby forming an end-of-use indicator.

10. The device as claimed in claim 1, in which said removable protective cap is connected to said dispensing head in a captive manner by a connector.

11. The device as claimed in claim 1, in which said removable protective cap comprises a first use indicator, secured to said dispensing head, and which is detached from said removable protective cap when the latter is removed from said dispensing head.

12. The device as claimed in claim 1, in which said removable protective cap comprises at least one axial tab which, in the closed position, penetrates into the interior of said dispensing head, each axial tab comprising a snap-fit projection co-operating with a portion of said dispensing head in order to prevent the removal of said cap, each axial tab being locked in its snap-fitted position by an axial extension of said pump body, which prevents any deformation of said axial tab, such that when said dosing chamber is filled, locking of said axial tab by said axial extension is eliminated by axial displacement of said dispensing head with respect to said pump body, enabling said cap to be removed.

13. A method for priming a device for dispensing fluid product, comprising the following steps:
providing a reservoir containing at least one dose of fluid product,
providing a dispenser comprising a pump body containing a dosing chamber, and a dispensing head comprising a dispensing orifice provided with a removable protective cap forming a closure, said dispensing head being axially displaceable with respect to said pump body, a piston, which is cohesive with said dispensing head, being adapted to slide in said dosing chamber between a high abutment position in which the volume of said dosing chamber is maximal, and a low abutment position, in which the volume of said dosing chamber is minimal,
characterized in that said method comprises the following steps:
fitting said dispenser on said reservoir, by inserting said pump body into said reservoir with said removable protective cap in the closed position and said piston in the low abutment position,
axially displacing said piston only once between its low abutment position and its high abutment position, in order to fill said dosing chamber from said reservoir,
removing said removable protective cap, and
axially displacing said piston only once between its high abutment position and its low abutment position, in order to dispense one dose or more doses of fluid product through said dispensing orifice.

14. The method as claimed in claim 13, in which said reservoir contains at least two doses of fluid which are loaded into the dosing chamber during the axial displacement of said piston between its low abutment position and its high abutment position, said device comprising dose separation means for stopping the actuating stroke of said piston before said low abutment position, in order to dispense said fluid product in at least two doses.
